(19) United States
(12) Patent Application Publication
(10) Pub. No.: US 2024/0189052 A1
(43) Pub. Date: Jun. 13, 2024

(54) APPARATUS, COMPUTER-IMPLEMENTED METHOD AND COMPUTER PROGRAM

(71) Applicant: Precision Robotics Limited, Londone (GB)

(72) Inventors: Malgorzata Kamedula, London (GB); Zhang Lin, Shenzhen, Guangdong (CN); Jindong Liu, Isleworth (GB)

(21) Appl. No.: 18/286,839

(22) PCT Filed: Apr. 12, 2022
(86) PCT No.: PCT/GB2022/050906

§ 371 Date: Oct. 13, 2023

(30) Foreign Application Priority Data
Apr. 14, 2021 (GB) 2105309.5

(51) Int. Cl. A61B 34/37; A61B 34/00; A61B 34/10; A61B 34/20; A61B 90/00; B25J 9/16

(52) U.S. Cl. CPC A61B 34/37; A61B 34/20; A61B 34/25; A61B 34/74; A61B 90/03; B25J 9/1689; A61B 2034/107; A61B 2562/0257

(57) ABSTRACT

An apparatus including: at least one processor; and at least one memory including computer program code, the at least one memory and computer program code configured to, with the at least one processor, cause the apparatus to: receive a recalibration command from a passive controller configured to remotely control a robotic instrument, wherein the passive controller and robotic instrument have freedom of movement within respective control and instrument workspaces, and wherein the control workspace is mapped to the instrument workspace to allow the position of the robotic instrument to track the position of the passive controller as the passive controller moves within the control workspace; and recalibrate the mapping of the control workspace to the instrument workspace in response to the recalibration command such that the current position of the passive controller corresponds to the current position of the robotic instrument.

### CROSS-REFERENCE TO RELATED APPLICATIONS

This Application is a National Stage Application under 35 U.S.C. § 371 of PCT Application No. PCT/GB2022/050906, filed Apr. 12, 2022 which claims priority to UK Patent Application No. GB2105309.5, filed Apr. 14, 2021, the entire contents of which are incorporated by reference herein.

### TECHNICAL FIELD

The invention relates generally to remotely controlled robotic systems and, in particular, an apparatus, associated method and computer program for recalibrating the tracking of a robotic instrument to a passive controller.

### BACKGROUND

Remotely controlled robotic systems have use in a variety of applications, particularly applications in which a human's access, safety or both are limited. For example, remotely controlled robotic systems are used in minimally invasive surgeries in which access to the site to be operated on is limited to natural cavities and/or small incisions. A human's hands are too large to access such areas and therefore small robots may be used instead which are controlled remotely by a surgeon. Remote controlled robotic systems are also used in military applications such as bomb disposal where a robot may be operated remotely from a safe distance.

From herein the invention is primarily described in relation to surgical robotic systems. However, this is for demonstrative purposes only and is not to the exclusion of the invention's application in other fields.

Known remotely controlled surgical robotics systems comprise a controller and a robotic instrument wherein a user may issue commands to the robotic instrument by manipulating the controller. Known controllers comprise a base, an articulatable arm coupled to the base and comprising a plurality of joints (e.g. rotatable joints or prismatic joints), and a handle coupled to the articulatable arm and movable relative to the base through motion of the joints. Known robotic instruments comprise a base, an actuatable arm coupled to the base and comprising a plurality of joints, and an end effector coupled to the actuatable arm and actuatable relative to the base through motion of the joints.

The controller and the robotic instrument, particularly the handle and end effector respectively, each have freedom of movement within respective control and instrument workspaces. Further, the handle and end effector may each have six degrees of freedom of movement in its respective workspace including translational movement along three axes and rotational movement about three axes. The handle and end effector may be considered as having a position in the respective workspace dependent upon translational movement and an orientation relative to the respective workspace dependent upon rotational movement. Further, the position and orientation of the handle or end effector may be considered, in combination, as the pose of the handle or end effector.

A user may manipulate the position and orientation of the handle within the controller workspace and the manipulations may be tracked via measurement of the motion of each joint in the articulatable arm. The manipulations may be converted to commands for the robotic instrument, particularly the joints of the actuatable arm, to actuate so that the position and orientation of the end effector moves in the instrument workspace correspondingly to the manipulations of the handle in the controller workspace.

Known controllers are active controllers meaning that, when the robotic system is in use, torque required to rotate each joint in the controller is actively varied depending on input from a user. For example, if the user were to let go of the controller, torque in each of the joints may be varied so that the handle is held in the last position and orientation that the user manipulated it to. In other words, the controller freezes unless the user provides further input. By extension, the robotic instrument freezes also.

Further, in instances where movement of the robotic instrument is limited by its available workspace, then a corresponding limitation may be enforced on the controller by limiting the rotation of joints beyond a certain point for example. This prevents the controller from ever becoming misaligned from the robotic instrument.

However, actively varying the torque of each joint in the controller requires the controller to comprise expensive and bulky components such as servo motors. Known active controllers are therefore expensive to manufacture and lack portability. Further, the active variation of torque in joints of the controller can encourage the user towards unwanted and/or unnatural positions which may frustrate the user or cause errors to be made.

The listing or discussion of a prior-published document or any background in this specification should not necessarily be taken as an acknowledgement that the document or background is part of the state of the art or is common general knowledge. One or more aspects/embodiments of the present disclosure may or may not address one or more of the background issues.

### SUMMARY

According to a first aspect of the invention, there is provided an apparatus comprising: at least one processor; and at least one memory including computer program code, the at least one memory and computer program code configured to, with the at least one processor, cause the apparatus to: receive a recalibration command from a passive controller configured to remotely control a robotic instrument, wherein the passive controller and robotic instrument have freedom of movement within respective control and instrument workspaces, and wherein the control workspace is mapped to the instrument workspace to allow the position of the robotic instrument to track the position of the passive controller as the passive controller moves within the control workspace; and recalibrate the mapping of the control workspace to the instrument workspace in response to the recalibration command such that the current position of the passive controller corresponds to the current position of the robotic instrument.

A passive controller is a controller comprising joints which are freely moveable with no active variation of the torque required to rotate the joints as there is in active controllers. A passive controller may be advantageous over known active controllers in that they may be cheaper to manufacture, smaller and lighter by virtue of not requiring expensive, heavy and or bulky components such as servo motors. Further, as there is no active variation of joint torque, there is no biasing of the user's movements towards uncomfortable or unnatural poses that may result in sub-optimal or unintended commands being transmitted to the associated robotic instrument.

However, as the user of a passive controller is free to manipulate the passive controller within its workspace with no artificial restriction, it is possible that the passive controller is moved to a position which causes misalignment from the associated robotic instrument. For example, the passive controller may be moved to a position in the control workspace that the robotic instrument is not able to replicate in the instrument workspace due to differences in the control and instrument workspaces. Also, the speed with which the robotic instrument can move within its workspace may be limited, for safety reasons for example, and the user of the passive controller may move the passive controller in the control workspace too quickly for the robotic instrument to mirror within the instrument workspace.

If the position of the passive controller becomes misaligned with the position of the robotic instrument, the user may struggle to continue controlling the robotic instrument accurately as movements of the passive controller in the control workspace may no longer be mirrored accurately by the robotic instrument in the instrument workspace. In other words, when the position of the passive controller becomes misaligned with the position of the robotic instrument, controlling the robotic instrument may become less intuitive.

By means of the present invention, mapping of the control workspace relative to the instrument workspace may be recalibrated so that the current position of the passive controller corresponds to the current position of the robotic instrument. Hence the position of the passive controller is re-aligned with the position of the robotic instrument and the user may more easily control the robotic instrument accurately.

In embodiments of the invention, the passive controller may comprise a clutch mechanism configured to enable the position of the passive controller within the control workspace to be changed without causing a corresponding change in the position of the robotic instrument within the instrument workspace, and the recalibration command may be received from the passive controller when the clutch mechanism is engaged or subsequently disengaged.

In such embodiments of the invention, a user of the passive controller may engage the clutch mechanism to stop translational movements of the passive controller within the control workspace causing corresponding translational movements of the robotic instrument in the instrument workspace. In other words, while the clutch mechanism is engaged, the robotic instrument will hold the last position it was actuated to prior to engagement of the clutch mechanism, even if the position of the passive controller is changed during that time.

This may be advantageous in instances where the user of the passive controller wants to move the controls to a more comfortable or easily accessible position in the control workspace but keep the robotic instrument in the same position within the instrument workspace. The clutch mechanism may also allow the user to take a break from controlling the robotic instrument. For example, during a long surgical procedure the surgeon may want to take a break to relax muscles which are being used to carry out very precise movements or to pass information or instructions to other members of the surgical team.

By virtue of the passive controller being moveable in the control workspace while the robotic instrument is stationary in the instrument workspace, it is likely that the passive controller will become misaligned with the robotic instrument during engagement of the clutch mechanism. However, recalibrating the mapping of the control workspace to the instrument workspace when the clutch mechanism is engaged/disengaged re-aligns the passive controller position with the robotic instrument position. This means that any changes the user has made to the position of the passive controller while the clutch mechanism has been engaged should have little to no effect on the accuracy and intuitiveness with which the user may control the robotic instrument once the clutch mechanism is disengaged.

In embodiments of the invention, the passive controller may comprise an engagement mechanism configured to initiate the tracking of the robotic instrument position to the passive controller position, and the recalibration command may be received from the passive controller on activation of the engagement mechanism.

In such embodiments of the invention, when operation of the robotic instrument is first initiated by a user, the control workspace may be mapped to the instrument workspace so that the current position of the passive controller corresponds to the starting position of the robotic instrument. Hence, the engagement mechanism ensures that the passive controller and robotic instrument are aligned when the user starts controlling the robotic instrument so that the user may control the passive controller accurately and intuitively.

In embodiments of the invention, the passive controller may comprise an unlock mechanism configured to reinitiate the tracking of the robotic instrument position to the passive controller position following a tracking interruption, and the recalibration command may be received from the passive controller on activation of the unlock mechanism.

In such embodiments of the invention, if operation of the robotic instrument is interrupted and the user is re-initiating operation, the passive controller and robotic instrument may be aligned by virtue of the unlock mechanism similarly to the engagement mechanism.

In embodiments of the invention, the orientation of the robotic instrument may track the orientation of the passive controller, and the apparatus may be configured to automatically control the orientation of the robotic instrument such that it is aligned with the orientation of the passive controller on activation of the engagement or unlock mechanisms.

When operation of the robotic instrument is first initiated or re-initiated after an interruption in the robotic instrument tracking the passive controller, it is likely that the orientation of the robotic instrument relative to the instrument workspace is misaligned with orientation of the passive controller relative to the control workspace. To remedy this, upon activation of the engagement or unlock mechanism, the apparatus may automatically control the orientation of the robotic instrument to align it with the current orientation of the passive controller.

When combined with recalibration of the mapping of the control workspace to the instrument workspace in order that the passive controller position corresponds to the robotic instrument position, both the position and the orientation of the passive controller and robotic instrument may be aligned. Accordingly, whenever the user initiates or reinitiates operation of the robotic instrument, they may start with the position and orientation of the robotic instrument being aligned with that of the passive controller. This may ensure that the user can control the robotic instrument comfortably and intuitively.

In embodiments of the invention, the apparatus may be configured to determine a trajectory of movement for the robotic instrument within the instrument workspace based on the current orientation of the passive controller to enable said automatic control.

In such embodiments of the invention, the trajectory may be determined to move the robotic instrument so that its orientation corresponds as closely as possible to that of the passive controller. Based on the trajectory of movement, commands may be issued to motors that drive actuation of the robotic instrument, thereby causing the robotic instrument to follow the trajectory of movement.

In embodiments of the invention, the apparatus may be configured to redetermine the trajectory of movement as the current orientation of the passive controller changes.

In such embodiments of the invention, in order that the orientation of the robotic instrument may be aligned with the current orientation of the passive controller, rather than an old and incorrect orientation of the passive controller, the trajectory of movement is updated based on the current orientation of the passive controller. Therefore there will be no misalignment of orientations caused by movement of the passive controller while the robotic instrument is being moved automatically.

In embodiments of the invention, the robotic instrument may be configured to be rearranged between an initial pose and one or more further poses, and the apparatus may be configured to automatically control the arrangement of the robotic instrument such that it returns from the one or more further poses to the initial pose on activation of a re-homing mechanism.

In such embodiments of the invention, the initial pose of the robotic instrument may be a combination of rotational positions of each joint forming part of the robotic instrument that is held when the robotic instrument is inactive. For example, the initial pose may correspond to a straight arrangement of joints which is advantageous for inserting or removing the robotic instrument from an operation site. The initial pose may also correspond to a neutral arrangement of joints from which the robotic instrument is readily moveable to any position and orientation within the instrument workspace.

The re-homing mechanism provides a means for automatically returning the robotic instrument to the initial pose from any other pose that the robotic instrument may have been moved to in use. This may be useful, for example, when a surgeon has completed a surgical procedure and is ready to withdraw the robotic instrument from the operation site or has completed a part of a surgical procedure and wishes to start the next stage of the procedure with the robotic instrument in a neutral arrangement.

In embodiments of the invention, the apparatus may be configured to limit the speed of movement of the robotic instrument to a predefined magnitude during alignment/arrangement of the robotic instrument.

In such embodiments of the invention, the automated movement of the robotic instrument may be limited to a speed that allows for suitable monitoring of the movement by the user and that reduces the risk of unsafe movements being made.

In embodiments of the invention, the apparatus may be configured to stop automatically controlling the robotic instrument on receipt of an override command or once the alignment/arrangement is complete.

In such embodiments of the invention, the user may monitor the automatic control until the robotic instrument has reached the required orientation or pose, at which point automatic control will stop and the user may resume control. If the user thinks that the trajectory which the robotic instrument is moving on during automatic control may be unsafe, the user may trigger the override command received by the apparatus. For example, the trajectory of the robotic instrument's movement determined by the apparatus may be moving the robotic instrument too close to a patient's soft tissue. The override command causes automatic control to stop so that the user may resume control of the robotic instrument. This may allow the user to navigate the robotic instrument away from the observed hazard, for example, and once it appears that the robotic instrument is in a safe position the interrupted alignment or arrangement process may be re-initiated by the user.

In embodiments of the invention, the engagement mechanism may comprise a proximity sensor configured to detect the presence or absence of a user, and the apparatus may be configured to initiate the tracking of the robotic instrument position to the passive controller position only when the proximity sensor has detected the presence of a user.

In such embodiments of the invention, the proximity sensor may be any suitable type of proximity sensor suitably configured to detect the presence or absence of a user. For example, the passive controller may comprise a handle held by the user when manipulating the passive controller. The proximity sensor may form part of the handle and may be configured to detect when the handle is being held by a user. The apparatus being configured to initiate tracking of the robotic instrument to the passive controller only when the proximity sensor detects the presence of a user may reduce risk of accidental movements of the passive controller causing a potentially dangerous corresponding movement of the robotic instrument.

In embodiments of the invention, the apparatus may be configured to stop the robotic instrument from tracking the position of the passive controller when the proximity sensor detects the absence of the user.

In such embodiments of the invention, no movements of the passive controller that follow the absence of the user being detected will be tracked by the robotic instrument. This may be particularly beneficial if, for example, the user mistakenly dropped the passive controller. As the passive controller is passive rather than active, it will move due to gravity if it is dropped by the user. In use during a surgical procedure, a corresponding movement of the robotic instrument could be harmful to the patient. Hence, the apparatus being configured to stop tracking of the robotic instrument to the passive controller when the absence of the user is detected may improve the safety of the robotic instrument.

In embodiments of the invention, the passive controller may comprise an electronic display screen configured to display a representation of the instrument and control workspaces, and the apparatus may be configured to control the electronic display screen such that the current position and/or orientation of the robotic instrument and passive controller are indicated within the representations of the respective instrument and control workspaces.

In such embodiments of the invention, the user may monitor the current position and/or orientation of the robotic instrument and passive controller within the respective instrument and control workspace as well as monitoring how the control workspace is mapped to the instrument workspace. This may help the user to understand degree of freedom and limitations to movement with which the robotic instrument is operable which may inform the user when it may be beneficial to initiate the unlock mechanism or re-homing mechanism. It may also help the user to understand when the passive controller becomes misaligned from the robotic instrument and when it may be necessary to engage the clutch mechanism, for example.

In embodiments of the invention, the apparatus may be configured to control the electronic display screen such that the current position and/or orientation are indicated in two or three dimensions.

In such embodiments of the invention, two-dimensional representation of the current position and/or orientation may be easier for the user to understand and may be particularly useful if the robotic instrument is limited to movement substantially in only two dimensions. Meanwhile, three-dimensional representation of the current position and/or orientation may be more informative to the user, particularly when the robotic instrument is free to move in three dimensions.

The position (e.g. on an x-y coordinate plane) of the robotic instrument and passive controller may be indicated by a point within the two-dimensional representations of the respective instrument and control workspaces. In addition, the orientation may be indicated by an arrow radiating from the point in two or three dimensions. Furthermore, a translational slider bar may be used to indicate the translational position (z) perpendicular to the x-y coordinate plane.

In embodiments of the invention, the current position and/or orientation of the passive controller and robotic instrument may be the last known position and/or orientation of the passive controller and robotic instrument to the apparatus, respectively.

In embodiments of the invention, the robotic instrument may comprise an end effector, and the current position and/or orientation of the robotic instrument may be the current position and/or orientation of the end effector.

In embodiments of the invention, the robotic instrument may be a surgical robotic instrument.

In embodiments of the invention, the apparatus may comprise the passive controller and/or robotic instrument.

According to a second aspect of the invention, there is provided a computer-implemented method comprising: receiving a recalibration command from a passive controller configured to remotely control a robotic instrument, wherein the passive controller and robotic instrument have freedom of movement within respective control and instrument workspaces, and wherein the control workspace is mapped to the instrument workspace to allow the position of the robotic instrument to track the position of the passive controller as the passive controller moves within the control workspace; and recalibrating the mapping of the control workspace to the instrument workspace in response to the recalibration command such that the current position of the passive controller corresponds to the current position of the robotic instrument.

The steps of any method disclosed herein do not have to be performed in the exact order disclosed, unless explicitly stated or understood by the skilled person.

According to a third aspect of the invention, there is provided a computer program (which may or may not be recorded on a carrier) comprising computer code configured to perform a method according to the second aspect of the invention.

The present disclosure includes one or more corresponding aspects, example embodiments or features in isolation or in various combinations whether or not specifically stated (including claimed) in that combination or in isolation. Corresponding means for performing one or more of the discussed functions are also within the present disclosure.

The above summary is intended to be merely exemplary and non-limiting.

### BRIEF DESCRIPTION OF THE FIGURES

Embodiments of the invention will now be described, by way of example only, with reference to the accompanying drawings, in which:

FIG. 1 is a schematic representation of an apparatus, according to the first aspect of the invention, in use;

FIG. 2 is a schematic representation of a passive controller forming part of the apparatus shown in FIG. 1;

FIG. 3 is a schematic representation of a robotic instrument forming part of the apparatus shown in FIG. 1;

FIG. 4 is a schematic representation of a control workspace mapped to an instrument workspace prior to recalibration;

FIG. 5 is a schematic representation of a control workspace mapped to an instrument workspace after recalibration;

FIG. 6 is a schematic representation of a clutch operation performable by the apparatus shown in FIG. 1;

FIG. 7 is a schematic representation of an engagement operation performable by the apparatus shown in FIG. 1;

FIG. 8 is a schematic representation of an unlock operation performable by the apparatus shown in FIG. 1;

FIG. 9 is a graphical representation of a global solution for an arbitrary function;

FIG. 10 is a graphical representation of local solutions for the arbitrary function shown in FIG. 9;

FIGS. 11, 12 and 13 are graphical representations of constrained and unconstrained solutions for different arbitrary functions;

FIG. 14 is a schematic representation of a re-homing operation performable by the apparatus shown in FIG. 1; and

FIG. 15 is a schematic representation of the operations shown in FIGS. 6 to 8 and 14 in combination.

### DESCRIPTION OF SPECIFIC ASPECTS/EMBODIMENTS

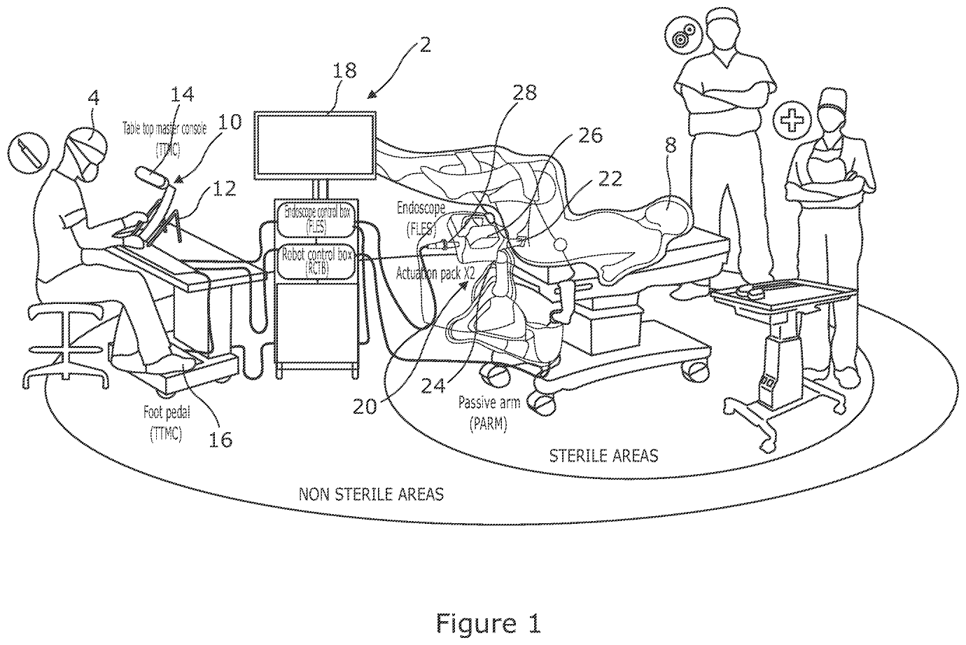

Referring initially to FIG. 1, an apparatus according to the first aspect of the invention is designated generally by the reference numeral 2. The apparatus 2 comprises at least one processor and at least one memory including computer program code. The apparatus 2 is shown being used by a user 4 in an operating theatre 6 to carry out a surgical procedure on a patient 8. The user 4 may therefore be a surgeon.

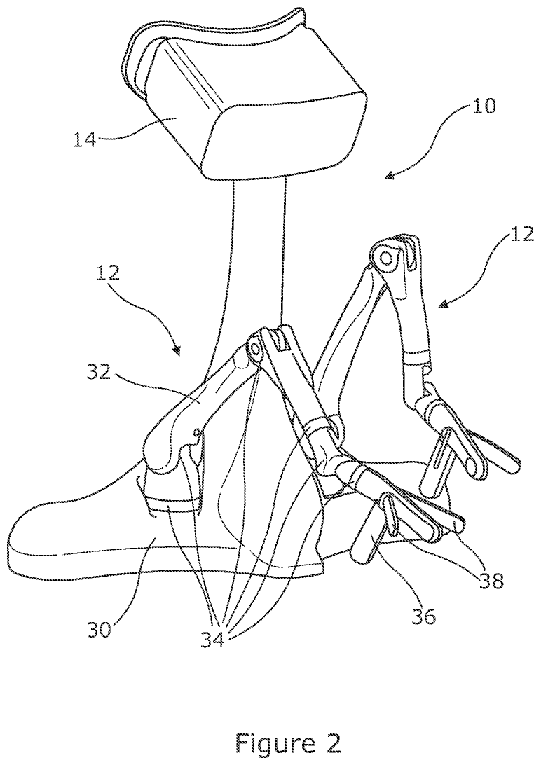

In this embodiment of the invention, the apparatus 2 further comprises a control station 10 including a pair of passive controllers 12 (shown in more detail in FIG. 2), a view port 14 and pedal controls 16. The passive controllers 12 may each be manipulated within respective control workspaces by the user 4.

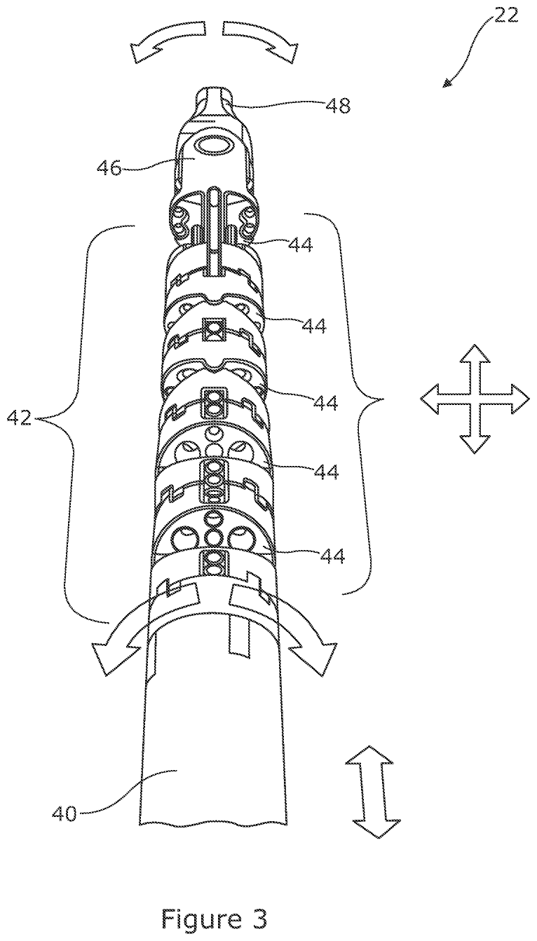

The apparatus further comprises a monitor 18 and a surgical robot 20 including a pair of robotic instruments 22 (one of which is shown in more detail in FIG. 3), a robot platform 24 and robot motors 26. The robotic instruments are mounted to the robot platform 24 which is arranged to position the robotic instruments inside the patient 8. The robot motors 26 are also mounted to the robot platform 24 and operatively coupled to the robotic instruments to drive movement of the robotic instruments within respective instrument workspaces.

The viewport 14 comprises an electronic display screen configured to display a representation of the instrument and control workspaces. Further, the apparatus 2 is configured to control the electronic display screen such that the current position and/or orientation of the robotic instruments and passive controllers 12 are indicated within the representations of the respective instrument and control workspaces. Also, the apparatus 2 is configured to control the electronic display screen 18 such that the current position and/or orientation are indicated in two or three dimensions.

The current position and/or orientation of the passive controller 12 and robotic instrument are the last known position and/or orientation of the passive controller 12 and robotic instrument to the apparatus, respectively.

An endoscope 28 is also mounted to the robot platform 24 and inserted inside the patient 9. The endoscope 28 may record images of site of the surgical procedure, including the robotic instruments in use, and the recorded images may be transmitted to the viewport 14, via the control station 10, to be displayed on the electronic display screen. The recorded images may be shown alongside the representations of the control and instrument workspaces, or with the workspaces superimposed on to the recorded images. The monitor 18 may display the recorded images, representations of the control and instrument workspaces, information on the status of the apparatus 2 or any suitable combination of these.

Referring now to FIG. 2, each passive controller 12 comprises a controller base 30; an articulatable arm 32, coupled to the controller base 30 and comprising a plurality of controller joints 34; and a handle 36, coupled to the articulatable arm 32 and comprising grippers 38. Each of the controller joints 34 are freely rotatable, i.e. there is no means for actively varying the torque required to rotate each joint as there would be in known active controllers. The handles 36 are thereby freely moveable within the respective control workspace by the user 4 (shown in FIG. 1). Further, each passive controller 12 and particularly each handle 36, may be considered as having a position in the respective control workspace and an orientation relative to the control workspace.

From here on embodiments of the invention are described with respect to a single passive controller 12 and a corresponding robotic instrument. However, it is to be understood that the apparatus may comprise two or more passive controllers and a corresponding number of robotic instruments.

Referring now to FIG. 3, a robotic instrument 22, equivalent to the robotic instrument positioned inside the patient 8 in FIG. 1, comprises a instrument base 40, an actuatable arm 42 and an end effector 46.

In this embodiment of the invention, the instrument base 40 is a shaft that may extend from the robot platform 24 shown in FIG. 1 and facilitate suitable positioning of actuatable arm 42 and end effector 46 relative to the patient 8. The actuatable arm 42 is coupled to the instrument base 40 and comprises a plurality of instrument joints 44. Each instrument joint 44 is rotatable and rotation may be driven by a motor forming part of the robot motors 26 shown in FIG. 1 via tendons that extend from the instrument platform 24, through the instrument base 40 and are attached to the relevant instrument joint 44. The end effector 46 is coupled to the actuatable arm 42 and is moveable within the instrument workspace through rotation of one or more of the instrument joints 44. The current position and/or orientation of the robotic instrument 22 comprises the current position and/or orientation of the end effector 46.

The end effector 46 also comprises a pair of jaws 48 moveable between an open configuration and a closed configuration (shown in FIG. 3). Opening and closing of the jaws 48 may be controlled by the user 4 by manipulating the grippers 38 of the corresponding passive controller 12 shown in FIG. 2.

The instrument workspace within which the end effector 46 is moveable is typically very different in scale to the control workspace within which the handle 36 is moveable. Further, the arrangement of the instrument joints 44 in the actuatable arm 42 is typically very different to the arrangement of the controller joints 34 in the articulatable arm 32 meaning that the shape of the instrument workspace often differs to the shape of the control workspace.

In use, the control workspace is mapped to the instrument workspace to allow the position of the robotic instrument 22 to track the position of the passive controller 12 as the passive controller 12 is moved within the control workspace by the user. However, the control workspace cannot always be mapped perfectly to the instrument workspace due to the differences described above. This means that is possible for the user 4 to move the handle 36 to a position in the control workspace which cannot be replicated by the end effector 46 moving within the instrument workspace.

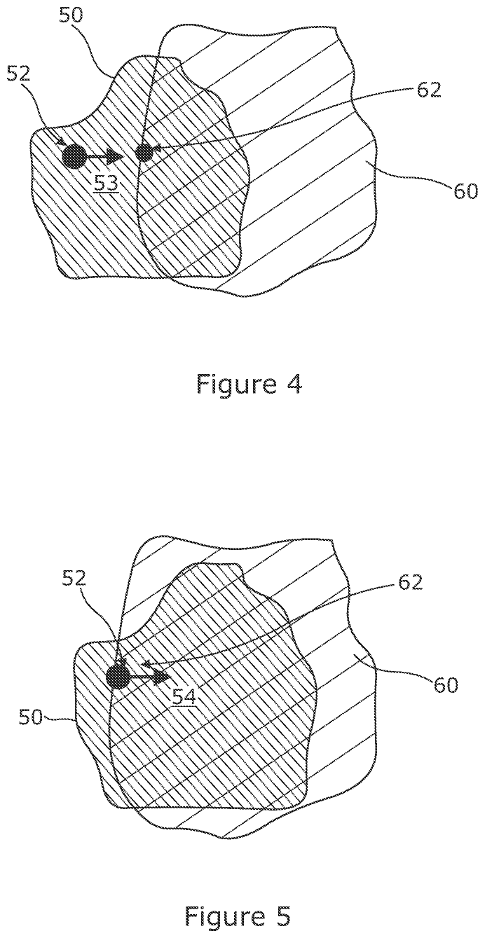

FIG. 4 shows a two-dimensional representation of a control workspace 50 mapped relative to an instrument workspace 60. In this example, the current position 52 of the passive controller in the control workspace 50 is outside of the instrument workspace 60, and the current position 62 of the robotic instrument in the instrument workspace 60 is as close to the current controller position 52 as possible while being within the limits of the instrument workspace 60.

In this situation, the user may move the controller position 52 right, towards the current instrument position 62 as indicated by the arrow 53, but the instrument will remain in the same position. It may therefore seem to the user 4 that the robotic instrument 22 has frozen.

The apparatus 2, particularly the at least one processor, memory and computer program code, is configured to receive a recalibration command from the user 4, via the passive controller 12, and recalibrate the mapping of the control workspace 50 to the instrument workspace 60 in response to the recalibration command such that the current position 52 of the passive controller 12 corresponds to the current position of the robotic instrument 12.

FIG. 5 shows the recalibrated mapping of the control workspace 50 to the instrument workspace wherein the controller position 52 corresponds to the current instrument position 62. Now, if the user 4 moves the passive controller 12 to the right as indicated by the arrow 54, a new instrument position may be calculated that corresponds to the new controller position and a trajectory for the robotic instrument 22 so that it moves towards the new instrument position and thereby tracks the controller position.

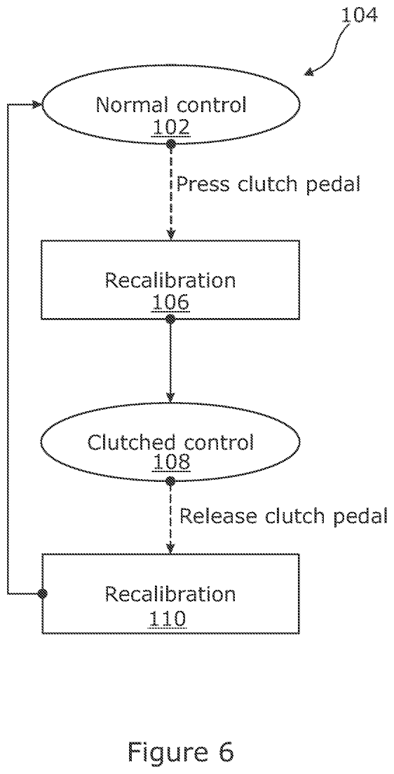

FIG. 6 shows how the recalibration of the control workspace 50 to the instrument workspace 60 may be incorporated into a clutch procedure 104 of the apparatus 2. Normal Control 102 represents the apparatus 2 in normal use when the robotic instrument 22 is tracking the position and orientation of the passive controller 12. The clutch procedure 104 may be initiated by the user 4 by pressing a clutch pedal.

Referring back to FIG. 1, the pedal controls 16 comprise a clutch mechanism (not shown) which the user 4 may engage to enable the position of the passive controller 12 within the control workspace to be changed without causing a corresponding change in the position of the robotic instrument 22 (shown in FIG. 3) within the instrument workspace. Therefore, engagement of the clutch mechanism disengages the robotic instrument 22 from tracking the position of the passive controller 12, but not necessarily the orientation. During clutched control, the orientation of the robotic instrument 22 may still change depending on the orientation of the passive controller 12.

In this embodiment of the invention, the clutch mechanism comprises a clutch pedal that may be pressed by the user 4 to engage the clutch mechanism and then released to disengage the clutch mechanism. However, in other embodiments of the invention, the clutch mechanism may comprise any suitable means for engagement and disengagement by a user such as a button, trigger, lever or voice command system for example.

In FIG. 6, when the clutch pedal is pressed, a recalibration command 106 is received and mapping of the control workspace 50 is recalibrated relative to the instrument workspace 60 as demonstrated in FIGS. 4 and 5. This ensures that the controller position 52 is aligned with the instrument position 62 prior to any movements of the passive controller 12 while the apparatus 2 is in a state of clutched control 108. Initial alignment of the controller position 52 with the instrument position 62 improves the quality of orientation tracking during clutched control 108. This is because commands causing movement of the robotic instrument to track the passive controller are computed so that the sum of the squared position and orientation tracking errors is minimized. When the position tracking error is significant (i.e., the gap between the controller position 52 and the instrument position 62), it may render the orientation tracking errors irrelevant for the overall tracking error. The algorithm for computing commands issued to the robotic instrument will therefore put more weight on minimizing/maintaining the position tracking error even if the orientation tracking errors increase as a result. However, recalibration reduces the position tracking error to zero by definition as the positions are now aligned. Thus, the orientation tracking error is the only one to be minimized, and the algorithm will have no incentive to allow the orientation tracking error to increase.

During clutched control 108, the instrument position 62 (shown in FIGS. 4 and 5) is no longer updated based on changes to the controller position 52 and hence the instrument position remains stationary. Meanwhile, the user 4 may move the passive controller 12 freely and the control position 52 will change accordingly, likely causing the control position 52 to become misaligned from the instrument position 62.

When clutched control 108 is ended by the user 4 releasing the clutch pedal, thereby disengaging the clutch mechanism, another recalibration command 110 is received so that mapping of the control workspace 50 is again recalibrated relative to the instrument workspace 60 as demonstrated in FIGS. 4 and 5. Accordingly, the apparatus 2 returns to normal control 102 and the user 4 may resume positional control of the robotic instrument 22 following disengagement of the clutch mechanism, as well as orientational control which was maintained throughout the clutch operation 104. Further, the new (post-clutch) control position 52 is aligned to the last known instrument position 62 determined prior to engaging the clutch mechanism. This avoids the user 4 struggling with misalignment after the clutch procedure 104 and also prevents the robotic instrument jerking or jumping to align with the control position 52 immediately after the clutch mechanism is disengaged.

During engagement of the clutch procedure 104, the user 4 maintains control of the orientation of the robotic instrument 22, particularly the end effector 46, by manipulating the orientation of the passive controller 12, particularly the handle 36. However, there are a number of situations wherein the user 4 may not have full control of the orientation of the robotic instrument 22, at least temporarily. For example, when the user 4 initiates use of the apparatus 2, the initial orientation of the robotic instrument 22 will likely be different to the orientation of the passive controller 12 being held by the user 4. For such situations, the apparatus 2 is configured to automatically control the orientation of the robotic instrument 22 such that it is aligned with the orientation of the passive controller 12.

During automatic control, the apparatus 2 determines a trajectory of movement for the robotic instrument 22 within the instrument workspace to move it from the current pose to the desired pose. However, as automatic control follows a recalibration, the primary purpose of the automatic control may be considered as moving the robotic instrument 22 from its current orientation to the desired orientation. The generated trajectories are sent to the robot motors 26 (shown in FIG. 1) which actuate the robotic instrument 22 as required. The apparatus 2 is configured to stop automatically controlling the robotic instrument 22 once the current pose reaches the desired pose such that the necessary alignment of the robotic instrument 22 is complete or on receipt of an override command. The override command may be initiated by the user 4 at any time during automatic control if the user 4 considers the current trajectory of the robotic instrument 22 to be potentially unsafe.

The apparatus 2 is configured to limit the speed of movement of the robotic instrument 22 to a predefined magnitude during automatic control for alignment of the robotic instrument 22. For example, the speed may be limited to a speed that allows the user 4 to monitor the trajectory of the robotic instrument 22 and ensure that the trajectory is not potentially unsafe.

As the speed at which the robotic instrument 22 moves during automatic control is limited, automatic control may not be completed instantaneously. There is therefore a chance that the user will manually change the position and orientation of the passive controller 12 as the robotic instrument 22 is under automatic control. To help avoid misalignment of the robotic instrument 22, the apparatus may be configured to redetermine the trajectory of movement as the position and orientation of the passive controller 12 changes.

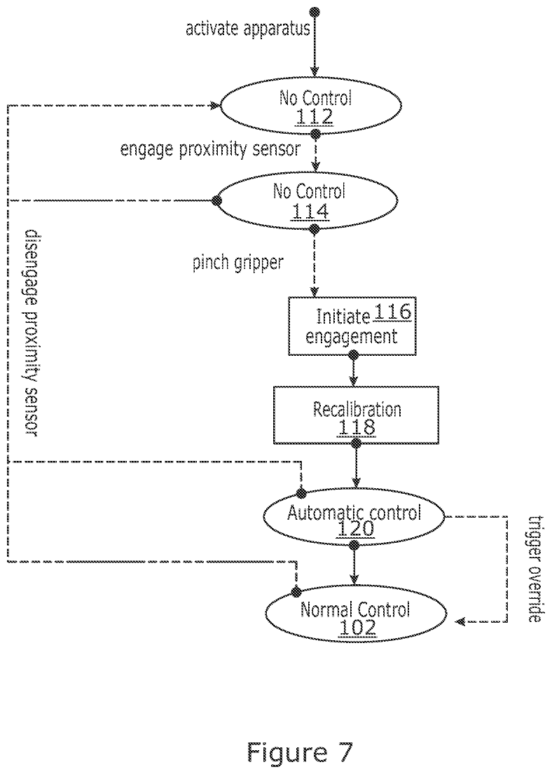

One example of when automatic control may be required is when the user 4 first takes control of the robotic instrument 22. In FIG. 7, when the apparatus 2 is activated it enters a first state of no control 112 wherein there is no tracking of the position or orientation of the robotic instrument 22 to that of the passive controller 12. In this embodiment of the invention, in order for the user 4 to take control of the robotic instrument 22 the user is required to initiate an engagement procedure 116.

The passive controller 12 comprises an engagement mechanism (not shown) comprising the grippers 38 (shown in FIG. 2) and a proximity sensor (not shown). The engagement mechanism is configured to initiate the tracking of the robotic instrument position and orientation to the passive controller position and orientation upon activation by the user 4.

The proximity sensor is configured to detect the presence or absence of a user, wherein the apparatus 2 is configured to initiate the tracking of the robotic instrument position to the passive controller position only when the proximity sensor has detected the presence of a user. Hence, a first step for the user to activate the engagement mechanism is to engage the proximity sensor and thereby enter the apparatus 2 into a second state of no control 114.

In this embodiment of the invention, the proximity sensor forms part of the handle 36 (shown in FIG. 2) and is configured to detect when the handle 36 is being held by the user 4. However, in other embodiments of the invention, the proximity sensor may be any suitable type of proximity sensor suitably configured to detect the presence or absence of a user.

A second step for the user 4 to activate the engagement mechanism is to pinch the grippers 38 (i.e. press and release) and thereby initiate the engagement procedure 116. This action demonstrates that the user 4 has full and intentional control of the passive controller 12.

In other embodiments of the invention, the engagement mechanism may comprise any suitable means for a user to initiate tracking of the robotic instrument to the passive controller such as a button, trigger, lever, pedal or voice command system for example. Further, the engagement mechanism may require a sequence of actions to initiate tracking of the robotic instrument to the passive controller such as a double tap or triple tap for example.

Upon initiation of the engagement procedure 116, a recalibration command 118 is received from the passive controller 12 to align the position of the passive controller 12 with the position of the robotic instrument 22. Then the apparatus 2 enters a state of automatic control 120 in which the apparatus 2 automatically controls the robotic instrument 22 to align its orientation with that of the passive controller as described above. Once the automatic control 120 has been completed or the user 4 triggers an override command, the apparatus 2 changes from automatic control 120 by the apparatus 2 to normal (manual) control 102 by the user.

The apparatus 2 is configured to stop the robotic instrument 22 from tracking the position and orientation of the passive controller 12 when the proximity sensor detects the absence of the user 4. Therefore, either during the second state of no control 114 or during automatic control 120 the engagement procedure 116 may be interrupted if the user 4 disengages the proximity sensor. Also, during normal control, the apparatus 2 will exit normal control 102 if the user 4 disengages the proximity sensor. When the proximity sensor is disengaged, the apparatus 2 will return to the first state of no control 112 until the proximity sensor is re-engaged to begin the engagement procedure 116 again.

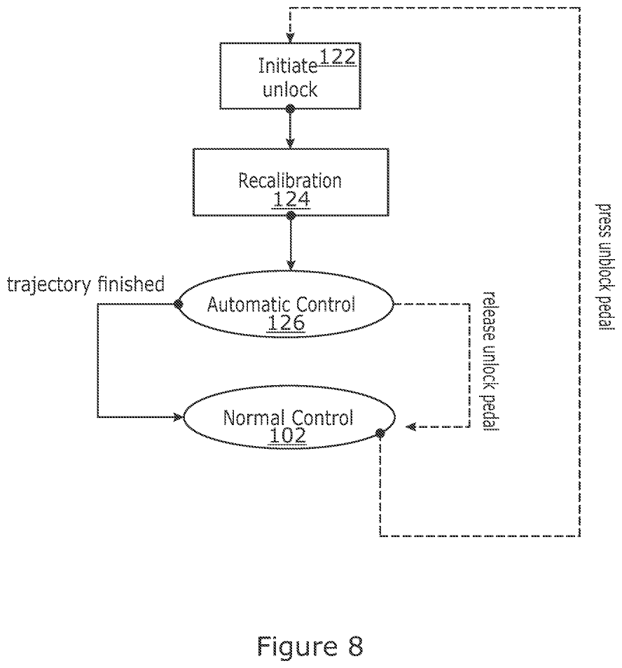

FIG. 8 shows an unlock procedure that may be used to reinitiate tracking of the position and/or orientation of the robotic instrument position to that of the passive controller following a tracking interruption. The user 4 may initiate the unlock procedure 122 for a number of different reasons. For example, the robotic instrument 22 may have frozen due to movement of the passive controller 12 by the user 4 being faster than the limited speed of the robotic instrument. On the other hand, the user may feel that the robotic instrument in a given configuration is out-of-control due to the apparatus 2 restricting the allowed poses of the robotic instrument.

The pedal controls 16 shown in FIG. 1 may comprise an unlock mechanism (e.g. an unlock pedal) that may be pressed/engaged by the user 4 to trigger the unlock procedure and then released/disengaged to terminate the unlock procedure. However, in other embodiments of the invention, the unlock mechanism may comprise any suitable means for engagement and disengagement by a user such as a button, trigger, lever or voice command system for example.

In FIG. 8, the user 4 may initiate the unlock procedure 122 by pressing the unlock pedal while the apparatus 2 is in normal control 102. When the unlock pedal is pressed, a recalibration command 124 is received from the passive controller 12 to align the position of the passive controller 12 with the position of the robotic instrument 22 as described previously. The apparatus 2 then enters a state of automatic control 126. During automatic control 126, the apparatus 2 generates a trajectory to align the pose of the robotic instrument 22 with the pose of the passive controller 12 as closely as possible. Although the positions of the robotic instrument 22 and passive controller 12 are aligned by the preceding recalibration, the determined trajectory may involve changing the position of the robotic instrument 22 if this improves the accuracy of its orientation with respect to the orientation of the passive controller 12 such that the overall pose of the robotic instrument is as close as possible to that of the passive controller.

In embodiments of the invention, the apparatus 2 uses an inverse kinematics (IK) algorithm to generate commands that cause the robotic instrument to track the pose of the passive controller.

Numerical IK algorithms can be divided into two subsets, global and local optimisation algorithms. The former look through the whole search space and provide the best possible solution (the global solution). The latter employs the knowledge of a mathematical function and looks for a best solution near the algorithm initial conditions, i.e., the initial state on which the further computations are based.

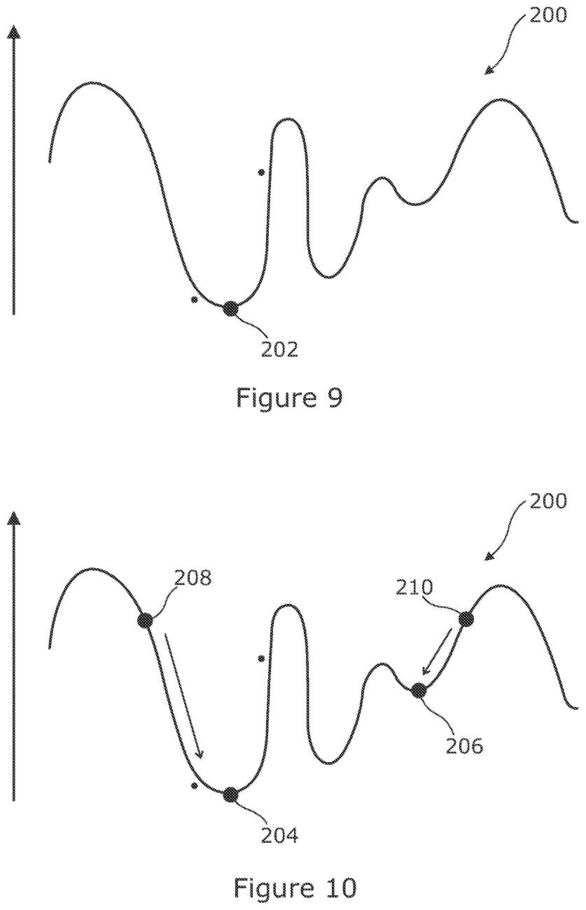

An example of a global solution 202, which may be found using a global algorithm, is shown in FIG. 9 for an arbitrary function 200; it is the overall minimum value of the function.

A local algorithm compares the current value of a function with its neighbourhood and moves the solution towards the smaller values. Further, a local algorithm terminates when all nearby values are higher than the current solution. FIG. 10 shows two possible local solutions 204, 206 for the same function 200 based on two different initial conditions 208, 210. One local solution 204 is equivalent to the global solution 202 (shown in FIG. 9) while the other local solution 206 is different.

If the apparatus 2 (shown in FIG. 1) were to use a local algorithm when the robotic instrument 22 is already in a local minimum with respect to the desired robotic instrument pose set by the user 4 with the passive controller 12, the local algorithm would keep returning the same local solution (e.g. 206 in FIG. 10) even though a better solution may be possible (e.g. 204 in FIG. 10). As a result, no motion of the robotic instrument will be performed and the robotic instrument 22 will seem unresponsive to the user 4.

Even though global algorithms may seem superior over local ones because they provide an objective best answer, local algorithms are often preferred because they are faster, terminate in a finite time and have better smoothness properties. For the purposes of this disclosure, the smoothness properties can be roughly interpreted as preventing the robotic instrument 22 from jumping or moving jerkily.

Embodiments of the invention find a global solution, while profiting from the efficacy of local algorithms, by implementing a random shooting approach on the initial conditions of a local algorithm. The local algorithm is restarted multiple times with randomly selected initial conditions. At the end, only the best local solution found is returned from the algorithm.

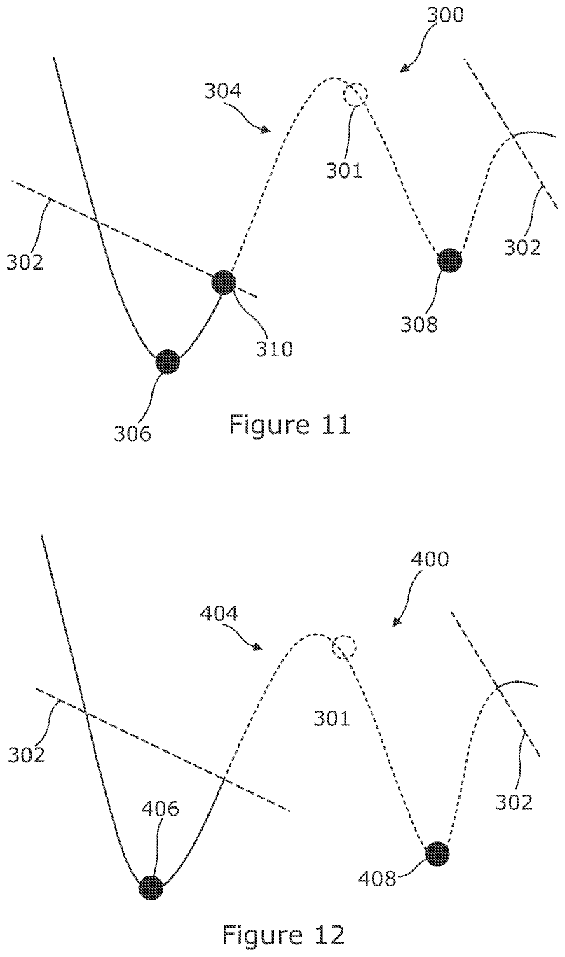

In use, the apparatus 2, and particularly the robotic instruments 22, are subject to various limitations. In particular, the position of the end effector 46 is limited to the instrument workspace. Also, the velocity at which the end effector 46 is allowed to move may be limited based on physical limitations of the robotic instrument 22 or a maximum velocity that is considered safe for a particular application. To account for these limitations, constraints are added to the optimisation problem that limit the IK algorithm to a set of feasible solutions. In FIG. 11, solutions are calculated for an arbitrary function 300 based on a current state 301. The feasible solutions are limited by constraints 302 to a constrained region 304 of the arbitrary function 300. An unconstrained global solution 306 exists outside of the constrained region 304 and is therefore not feasible. Meanwhile a local solution 308 exists within the constrained region 304 and is hence feasible. There is also a constrained global solution 310 which is feasible, as it is within the constrained region 304, and is also superior to the unconstrained local solution 308.

In FIG. 12, solutions are calculated for a new arbitrary function 400 based on the same current state 301 and with the same constraints 302 applied to provide a constrained region 404. In this example, an unconstrained global solution 406 exists similarly to that shown in FIG. 11 which is outside of the constrained region 404 and is therefore not feasible. However, for arbitrary function 400, a local solution 408 exists which is the best available solution in the constrained region 404 and is therefore the constrained global solution.

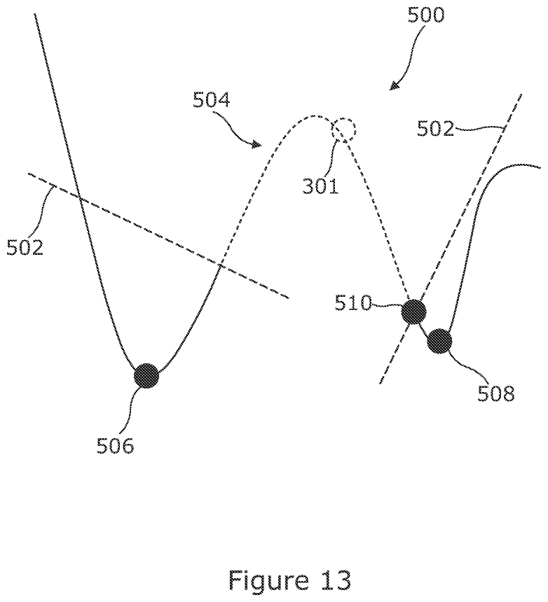

In FIG. 13, solutions are calculated for a further arbitrary function 500 based on the same current state 301 but with new constraints 502 applied providing a constrained region 504. An unconstrained global solution 506 exists similarly to that shown in FIGS. 11 and 12 which is outside of the constrained region 504 and is therefore not feasible. In this instance an unconstrained local solution 508 also exists outside of the constrained region 504. A constrained local solution 510 exists within the constrained region 504 which is the best available solution in the constrained region 504 and is therefore the constrained global solution as well as the constrained local solution.

In embodiments of the invention, the IK algorithm includes a predefined position limit to enable the calculation of partially constrained poses of the robotic instrument 22 that satisfy position constraints. The position constraints may be based on the instrument workspace which is, in turn, based on physical limitations of the robotic instrument 22 (FIG. 3) such as the length of the robotic instrument 22, the range of motion achievable by each joint 44 and the position of each joint 44 along the length of the robotic instrument 22. To ensure that any solution calculated by the IK algorithm is physically possible, the position constrains may always be applied when the apparatus 2 is in use such that all solutions are partially constrained.

The IK algorithm may further include a predefined velocity limit to enable the calculation of constrained poses of the robotic instrument 22 that satisfy both position and velocity constraints. The predefined velocity limit may be based on a maximum velocity for the robotic instrument 22 that is deemed safe and allows a user to monitor the movement, gauge the trajectory and have the ability to override the movement if it is potentially unsafe.

Under normal conditions, when the apparatus 2 is in use, the robotic instrument 22 will be restricted to constrained poses that satisfy both the position and velocity constraints. Under such conditions, the IK algorithm is used to calculate constrained global solutions i.e. the best possible solution with the constraints applied. However, the IK algorithm may simultaneously calculate partially constrained global solutions which are only limited with respect to the position constraints.

Activation of the unlock mechanism may cause the apparatus 2 to check if the robotic instrument's current constrained pose, based on the constrained global solution calculated by the IK algorithm, is equivalent to a partially constrained pose based on the simultaneously calculated partially constrained global solution, i.e. the best available solution within the position limits of the robotic instrument. If not, the apparatus will move the robotic instrument to the partially constrained pose.

To avoid sudden unexpected motions of the robotic instrument 22 when the unlock mechanism is activated, the automatic control may be limited in speed, as described earlier. When the determined trajectory is completed, the apparatus 2 then returns to normal control 102.

The unlock procedure 122 is running when the user is engaged with the passive controller 12, and thus, it is likely that the passive controller will be moved while the automatic control 126 is taking place. To avoid a mismatch between the position and/or orientation of the passive controller 12 and the robotic instrument 22, the determined trajectory is updated throughout the unlock procedure, as described previously. Furthermore, if the unlock pedal is released at any moment prior to the apparatus 2 returning to normal control 102, the apparatus 2 is immediately returned to normal (manual) control 102. Releasing the unlock pedal is therefore equivalent to triggering the override command as shown in FIG. 7.

In embodiments of the invention, the robotic instrument 22 may be configured to be rearranged between an initial pose and one or more further poses, and the apparatus 2 may be configured to automatically control the arrangement of the robotic instrument 22 such that it returns from the one or more further poses to the initial pose on activation of a re-homing mechanism.

In such embodiments, the initial pose of the robotic instrument 22 may be a combination of rotational positions of each joint 44 forming part of the robotic instrument 22 that is held when the robotic instrument 22 is inactive. For example, the initial pose may correspond to a straight arrangement of joints 44 (as shown in FIG. 3) which is advantageous for inserting or removing the robotic instrument 22 from an operation site. The initial pose may also correspond to a neutral arrangement of joints 44 from which the robotic instrument 22 is readily moveable to any position and orientation within the instrument workspace.

The re-homing mechanism provides a means for automatically returning the robotic instrument 22 to the initial pose from any other pose that the robotic instrument 22 may have been moved to in use. This may be useful, for example, when a surgeon has completed a surgical procedure and is ready to withdraw the robotic instrument 22 from the operation site or has completed a part of a surgical procedure and wishes to start the next stage of the procedure with the robotic instrument 22 in a neutral arrangement.

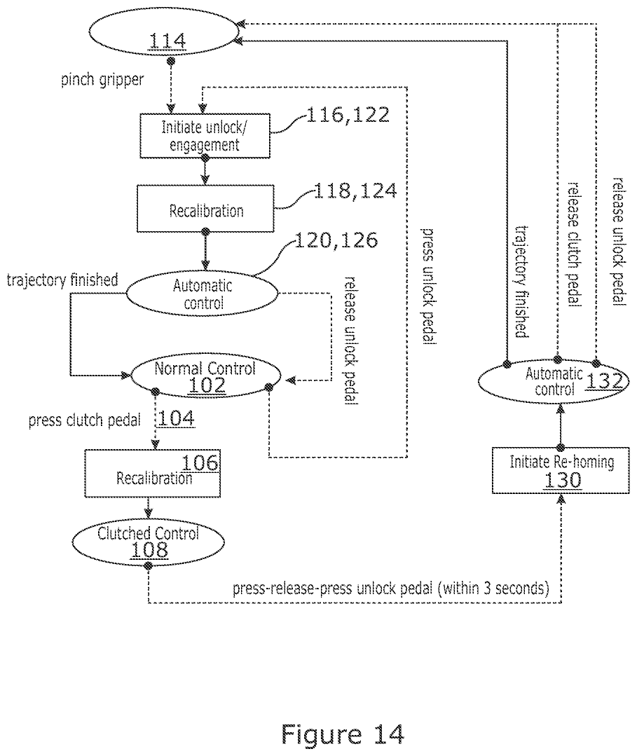

The re-homing mechanism may comprise the clutch pedal and the unlock pedal and may be activated by pressing the clutch pedal and unlock pedal in a predetermined sequence. In FIG. 14, the user 4 may initiate a rehoming procedure 130 by first pressing the clutch pedal. In accordance with the clutch procedure 104 shown in FIG. 6, pressing the clutch pedal will cause a recalibration command 106 to be received and mapping of the control workspace 50 to be recalibrated relative to the instrument workspace 60 as demonstrated in FIGS. 4 and 5. The apparatus 2 will then enter clutched control 108. While clutched control 108 is active, the user 4 may press release and press again the unlock pedal within a period of 3 seconds to initiate the re-homing procedure 130.

In other embodiments of the invention, the re-homing mechanism comprise any suitable means for activation by a user, such as a separate pedal, button, trigger, lever or voice command. Also, a different combination or sequence of the user interacting with other means such as the clutch pedal and unlock pedal may be used to initiate the re-homing procedure 130 and a different time period may be provided for the combination or sequence of actions to be taken.

Once the re-homing procedure 130 is initiated, the apparatus 2 begins automatic control 132 similar to the automatic control 120, 126 shown in FIGS. 7 and 8 except that the trajectory of movement for the robotic instrument 22 is determined to move the robotic instrument 22 to the initial pose. Furthermore, the apparatus 2 may be configured to limit the speed of movement of the robotic instrument 22 to a predefined magnitude when returning the robotic instrument 22 to the initial pose.

In contrast to the automatic control 120 shown in FIG. 7, once the trajectory is finished or an override command is triggered by the user 4 by releasing the clutch pedal or the unlock pedal, the apparatus 2 enters the second state of no control 114 shown in FIG. 7. In order for the user 4 to regain control of the robotic instrument, they must pinch the gripper 38 to initiate the engagement procedure 116 shown in FIG. 7. This is because during the arrangement of the robotic instrument towards the initial pose, the robotic instrument 22 may have become significantly misaligned from the passive controller 12.

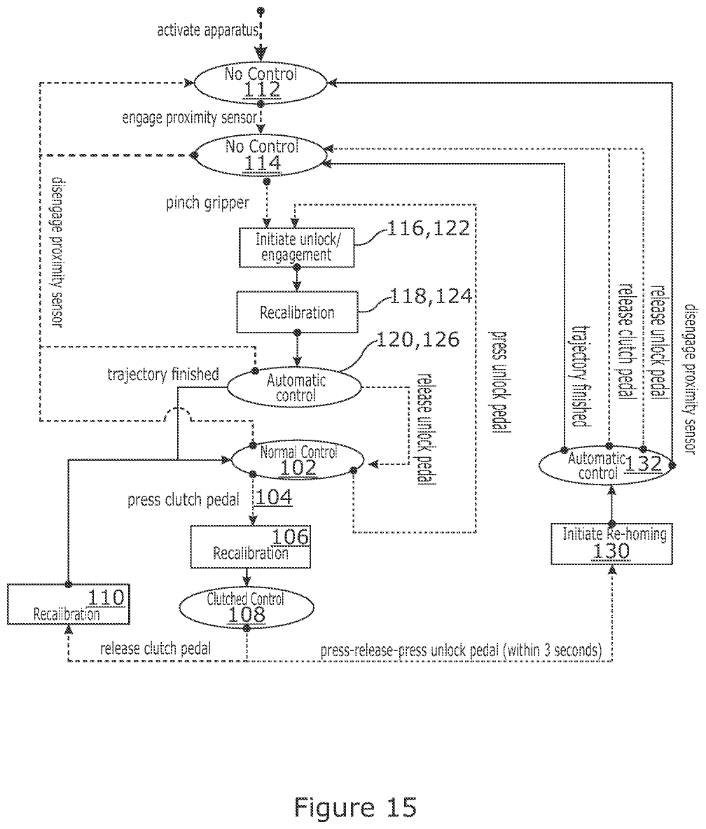

FIG. 15 shows one possible process flow which allows the apparatus 2 to perform each of the operations shown in FIGS. 6 to 8 and 14. In accordance with FIG. 7, the apparatus 2 is configured to stop the robotic instrument 22 from tracking the position and orientation of the passive controller 12 when the proximity sensor detects the absence of the user 4. Therefore, either during the second state of no control 114 or during automatic control 120/126, the engagement procedure 116/unlock procedure 122 may be interrupted if the user 4 disengages the proximity sensor. Also, during normal control, the apparatus 2 will exit normal control 102 if the user 4 disengages the proximity sensor. Additionally, during the re-homing procedure 130, the apparatus 2 will exit automatic control 132 if the user disengages the proximity sensor. In each case, when the proximity sensor is disengaged, the apparatus 2 will return to the first state of no control 112 until the proximity sensor is re-engaged to begin the engagement procedure 116 again.

The applicant hereby discloses in isolation each individual feature described herein and any combination of two or more such features, to the extent that such features or combinations are capable of being carried out based on the present specification as a whole, in the light of the common general knowledge of a person skilled in the art, irrespective of whether such features or combinations of features solve any problems disclosed herein, and without limitation to the scope of the claims. The applicant indicates that the disclosed aspects/embodiments may consist of any such individual feature or combination of features. In view of the foregoing description it will be evident to a person skilled in the art that various modifications may be made within the scope of the disclosure.

1. An apparatus comprising:
at least one processor; and
at least one memory including computer program code, the at least one memory and computer program code configured to, with the at least one processor, cause the apparatus to:
receive a recalibration command from a passive controller configured to remotely control a robotic instrument, wherein the passive controller and robotic instrument have freedom of movement within respective control and instrument workspaces, and wherein the control workspace is mapped to the instrument workspace to allow the position of the robotic instrument to track the position of the passive controller as the passive controller moves within the control workspace; and
recalibrate the mapping of the control workspace to the instrument workspace in response to the recalibration command such that the current position of the passive controller corresponds to the current position of the robotic instrument.

2. The apparatus of claim 1, wherein the passive controller comprises a clutch mechanism configured to enable the position of the passive controller within the control workspace to be changed without causing a corresponding change in the position of the robotic instrument within the instrument workspace, and wherein the recalibration command is received from the passive controller when the clutch mechanism is engaged or subsequently disengaged.

3. The apparatus of claim 1, wherein the passive controller comprises an engagement mechanism configured to initiate the tracking of the robotic instrument position to the passive controller position, and wherein the recalibration command is received from the passive controller on activation of the engagement mechanism.

4. The apparatus of claim 3, wherein the passive controller comprises an unlock mechanism configured to reinitiate the tracking of the robotic instrument position to the passive controller position following a tracking interruption, and wherein the recalibration command is received from the passive controller on activation of the unlock mechanism.

5. The apparatus of claim 4, wherein the orientation of the robotic instrument tracks the orientation of the passive controller, and wherein the apparatus is configured to automatically control the orientation of the robotic instrument such that it is aligned with the orientation of the passive controller on activation of the engagement or unlock mechanisms.

6. The apparatus of claim 5, wherein the apparatus is configured to determine a trajectory of movement for the robotic instrument within the instrument workspace based on the current orientation of the passive controller to enable said automatic control.

7. The apparatus of claim 6, wherein the apparatus is configured to redetermine the trajectory of movement as the current orientation of the passive controller changes.

8. The apparatus of claim 5, wherein the robotic instrument is configured to be rearranged between an initial pose and one or more further poses, and wherein the apparatus is configured to automatically control the arrangement of the robotic instrument such that it returns from the one or more further poses to the initial pose on activation of a re-homing mechanism.

9. The apparatus of claim 8, wherein the apparatus is configured to limit the speed of movement of the robotic instrument to a predefined magnitude during alignment/arrangement of the robotic instrument.

10. The apparatus of claim 9, wherein the apparatus is configured to stop automatically controlling the robotic instrument on receipt of an override command or once the alignment/arrangement is complete.

11. The apparatus of claim 3, wherein the engagement mechanism comprises a proximity sensor configured to detect the presence or absence of a user, and wherein the apparatus is configured to initiate the tracking of the robotic instrument position to the passive controller position only when the proximity sensor has detected the presence of a user.

12. The apparatus of claim 11, wherein the apparatus is configured to stop the robotic instrument from tracking the position of the passive controller when the proximity sensor detects the absence of the user.

13. The apparatus of claim 1, wherein the passive controller comprises an electronic display screen configured to display a representation of the instrument and control workspaces, and wherein the apparatus is configured to control the electronic display screen such that the current position and/or orientation of the robotic instrument and passive controller are indicated within the representations of the respective instrument and control workspaces.

14. The apparatus of claim 13, wherein the apparatus is configured to control the electronic display screen such that the current position and/or orientation are indicated in two or three dimensions.

15. The apparatus of claim 14, wherein the current position and/or orientation of the passive controller and robotic instrument are the last known position and/or orientation of the passive controller and robotic instrument to the apparatus, respectively.

16. The apparatus of claim 14, wherein the robotic instrument comprises an end effector, and wherein the current position and/or orientation of the robotic instrument is the current position and/or orientation of the end effector.

17. The apparatus of claim 1, wherein the robotic instrument is a surgical robotic instrument.

18. The apparatus of claim 1, wherein the apparatus comprises at least one of: the passive controller and robotic instrument.

19. A computer-implemented method comprising:
receiving a recalibration command from a passive controller configured to remotely control a robotic instrument, wherein the passive controller and robotic instrument have freedom of movement within respective control and instrument workspaces, and wherein the control workspace is mapped to the instrument workspace to allow the position of the robotic instrument to track the position of the passive controller as the passive controller moves within the control workspace; and
recalibrating the mapping of the control workspace to the instrument workspace in response to the recalibration command such that the current position of the passive controller corresponds to the current position of the robotic instrument.

20. A computer program comprising computer code which, when executed by a computer, causes the computer to perform the method of claim 19.
